(19) United States
(12) Patent Application Publication
(10) Pub. No.: US 2025/0076285 A1
(43) Pub. Date: Mar. 6, 2025

(54) TECHNIQUES FOR SCREENING CELLULAR TOXICITY

(71) Applicant: The Joan and Irwin Jacobs Technion-Cornell Institute, New York, NY (US)

(72) Inventors: R. Lea SANFORD, New York, NY (US); Helgi Ingolfur INGOLFSSON, Newark, CA (US); Radda RUSINOVA, New York, NY (US); Thasin PEYEAR, New York, NY (US); Olaf Sparre ANDERSEN, Old Chatham, NY (US)

(73) Assignee: THE JOAN AND IRWIN JACOBS TECHNION-CORNELL INSTITUTE, NEW YORK, NY (US)

(21) Appl. No.: 18/818,881

(22) Filed: Aug. 29, 2024

Related U.S. Application Data
(60) Provisional application No. 63/579,716, filed on Aug. 30, 2023.

(51) Int. Cl. G01N 33/50

(52) U.S. Cl. CPC G01N 33/5076

(57) ABSTRACT

Techniques for screening. A lipid and gramicidin are mixed and prepared for processing. The mixture is frozen and then thawed in a series of cycles before being extruded. The extruded mixture is frozen and thawed. Once thawed, the extruded mixture may be desalted to provide a liposome solution. The liposome solution may be stored for subsequent use such as, but not limited to, for use in screening tests.

### CROSS-REFERENCE TO RELATED APPLICATIONS

This application claims the benefit of U.S. Provisional Patent Application No. 63/579,716 filed on Aug. 30, 2023, the contents of which are hereby incorporated by reference.

### TECHNICAL FIELD

The present disclosure relates generally to cellular screening, and more specifically to techniques for preparing mixtures for screening in order to improve stability for screening purposes.

### BACKGROUND

Modern drug development often uses cellular screening for drug lead discovery. A significant factor in cellular screening is the effects that changes in biological membranes, particularly the lipid bilayer, can have on biological functions. In this regard, it is noted that the lipid bilayer component of biological membranes is involved in regulation of membrane protein function. Consequently, fluctuations in properties of the lipid bilayer can affect numerous biological functions.

Biological membranes to be tested may be prepared in different ways. In particular, some screening processes leverage synthetic membranes which may be designed and prepared in different ways depending on desirable characteristics for screening purposes. As a result, techniques which provide new ways to prepare membranes, and in particular to improve membrane preparation for screening of various materials, are highly desirable.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for solution preparation. The method comprises: mixing a lipid and a protein to create a mixture; freezing and thawing the mixture in a plurality of cycles; extruding the mixture after the plurality of cycles; and producing a vesicle solution using the extruded mixture.

Certain embodiments disclosed herein include a method, non-transitory computer readable medium, or system as noted above or below, wherein the protein is a transmembrane protein.

Certain embodiments disclosed herein include a method, non-transitory computer readable medium, or system as noted above or below, wherein the protein is gramicidin.

Certain embodiments disclosed herein include a method, non-transitory computer readable medium, or system as noted above or below, further including or being configured to perform the following step or steps: freezing the extruded mixture; and thawing the frozen extruded mixture, wherein the vesicle solution is produced using the extruded mixture when the extruded mixture has been frozen and thawed.

Certain embodiments disclosed herein include a method, non-transitory computer readable medium, or system as noted above or below, further including or being configured to perform the following step or steps: running the extruded mixture through a desalting column.

Certain embodiments disclosed herein include a method, non-transitory computer readable medium, or system as noted above or below, further including or being configured to perform the following step or steps: mixing a lipid solubilized in a first organic solvent and a gramicidin solubilized in a second organic solvent to create a film; and drying the film, where the mixture includes the film after the film has been dried.

Certain embodiments disclosed herein include a method, non-transitory computer readable medium, or system as noted above or below, further including or being configured to perform the following step or steps: rehydrating the film when the film has been dried, wherein the mixture includes the film which has been rehydrated.

Certain embodiments disclosed herein include a method, non-transitory computer readable medium, or system as noted above or below, wherein the film is rehydrated for at least 4 hours.

Certain embodiments disclosed herein include a method, non-transitory computer readable medium, or system as noted above or below, wherein the film is rehydrated for between 1 and 2 hours.

Certain embodiments disclosed herein include a method, non-transitory computer readable medium, or system as noted above or below, further including or being configured to perform the following step or steps: screening, using the vesicle solution, for at least one of cytotoxicity, toxicity, clinical trial failure, cosmetic ingredients, skincare ingredients, drug delivery systems, food additives, supplements, nutraceuticals, molecules, biologics, and environment toxins.

Certain embodiments disclosed herein include a method, non-transitory computer readable medium, or system as noted above or below, wherein the lipid is a lipid mixture of 1,2-dierucoyl-sn-glycero-3-phosphocholine (DC22:1PC).

Certain embodiments disclosed herein include a method, non-transitory computer readable medium, or system as noted above or below, wherein vesicle solution includes a gramicidin mixture of between 80 and 85 percent gramicidin A.

Certain embodiments disclosed herein include a method, non-transitory computer readable medium, or system as noted above or below, further including or being configured to perform the following step or steps: treating the vesicle solution using a test substance; incubating the vesicle solution which has been treated with the test substance; exposing the vesicle solution to a quencher solution; and recording a plurality of signals for the vesicle solution.

Certain embodiments disclosed herein include a method, non-transitory computer readable medium, or system as noted above or below, further including or being configured to perform the following step or steps: determining a specificity of the drug based on the recorded signals.

Certain embodiments disclosed herein include a method, non-transitory computer readable medium, or system as noted above or below, wherein the plurality of signals is recorded after a period of exposure time, further including or being configured to perform the following step or steps: determining a dose-response relationship for the period of exposure time.

Certain embodiments disclosed herein include a method, non-transitory computer readable medium, or system as noted above or below, wherein the quencher solution includes gramicidin channel permeable cations.

Certain embodiments disclosed herein include a method, non-transitory computer readable medium, or system as noted above or below, wherein the quencher solution includes a monovalent cation of a pair of an indicator and the monovalent cation.

Certain embodiments disclosed herein include a method, non-transitory computer readable medium, or system as noted above or below, wherein the vesicle solution includes a plurality of vesicles, further including or being configured to perform the following step or steps: analyzing the plurality of signals; and determining an effect of the test substance on lipid bilayers of the plurality of vesicles of the vesicle solution.

Certain embodiments disclosed herein include a method, non-transitory computer readable medium, or system as noted above or below, wherein each of the vesicles has a diameter between 100 and 400 nanometers.

Certain embodiments disclosed herein include a method, non-transitory computer readable medium, or system as noted above or below, wherein each of the vesicles has a diameter of 130 nanometers.

Certain embodiments disclosed herein include a method, non-transitory computer readable medium, or system as noted above or below, wherein each of the vesicles has a diameter of 300 nanometers.

Certain embodiments disclosed herein include a method, non-transitory computer readable medium, or system as noted above or below, wherein the determined effect of the test substance includes at least one change in membrane property.

Certain embodiments disclosed herein include a method, non-transitory computer readable medium, or system as noted above or below, further including or being configured to perform the following step or steps: determining a viability of a drug candidate for at least one purpose based on at least one change in membrane property.

Certain embodiments disclosed herein include a method, non-transitory computer readable medium, or system as noted above or below, wherein the determined effect of the test substance includes a lack of change in membrane properties.

Certain embodiments disclosed herein include a method for screening. The method comprises: treating a plurality of vesicles using a solution of at least one test substance, wherein at least a portion of the plurality of vesicles is created by at least premixing a lipid and a protein into a mixture before freezing and thawing the mixture; incubating the plurality of vesicles; exposing the plurality of vesicles to a quencher solution; and recording a plurality of signals for the plurality of vesicles.

Certain embodiments disclosed herein include a method, non-transitory computer readable medium, or system as noted above or below, wherein the quencher solution includes gramicidin channel permeable cations.

Certain embodiments disclosed herein include a method, non-transitory computer readable medium, or system as noted above or below, wherein the quencher solution includes a monovalent cation of a pair of an indicator and the monovalent cation.

Certain embodiments disclosed herein include a method, non-transitory computer readable medium, or system as noted above or below, wherein the plurality of signals is a plurality of fluorescence signals of fluorophores in the plurality of vesicles.

Certain embodiments disclosed herein include a method, non-transitory computer readable medium, or system as noted above or below, wherein the plurality of vesicles is a first plurality of vesicles included in a batch, wherein the batch includes the first plurality of vesicles and a second plurality of vesicles, further including or being configured to perform the following step or steps: comparing the plurality of signals to a baseline of the second plurality of vesicles.

Certain embodiments disclosed herein include a method, non-transitory computer readable medium, or system as noted above or below, wherein the second plurality of vesicles is not exposed to the test substance.

Certain embodiments disclosed herein include a method, non-transitory computer readable medium, or system as noted above or below, wherein the second plurality of vesicles is treated using a different concentration of the test substance than the first plurality of vesicles.

Certain embodiments disclosed herein include a method, non-transitory computer readable medium, or system as noted above or below, further including or being configured to perform the following step or steps: determining an effect of the test substance on the first plurality of vesicles based on the plurality of signals.

Certain embodiments disclosed herein include a method, non-transitory computer readable medium, or system as noted above or below, wherein the plurality of vesicles is from a vesicle solution produced using a lipid-gramicidin mixture.

Certain embodiments disclosed herein include a method, non-transitory computer readable medium, or system as noted above or below, wherein the lipid-gramicidin mixture is produced using a process including premixing lipid and gramicidin into a film before drying the film to produce a dried film.

Certain embodiments disclosed herein include a method, non-transitory computer readable medium, or system as noted above or below, wherein the dried film is rehydrated, wherein the lipid-gramicidin mixture includes the rehydrated film.

Certain embodiments disclosed herein include a method, non-transitory computer readable medium, or system as noted above or below, wherein the plurality of vesicles is treated using the solution of the test substance at a predetermined concentration.

Certain embodiments disclosed herein include a method, non-transitory computer readable medium, or system as noted above or below, wherein each of the vesicles has a diameter between 100 and 400 nanometers.

Certain embodiments disclosed herein include a method, non-transitory computer readable medium, or system as noted above or below, wherein each of the vesicles has a diameter of 130 nanometers.

Certain embodiments disclosed herein include a method, non-transitory computer readable medium, or system as noted above or below, wherein each of the vesicles has a diameter of 300 nanometers.

Certain embodiments disclosed herein include a method, non-transitory computer readable medium, or system as noted above or below, further including or being configured to perform the following step or steps: emitting light on the plurality of vesicles in order to excite the plurality of vesicles; and measuring the plurality of signals based on the excited vesicles.

Certain embodiments disclosed herein include a method, non-transitory computer readable medium, or system as noted above or below, further including or being configured to perform the following step or steps: distributing the plurality of vesicles among a plurality of plates, wherein the light is emitted on the plurality of vesicles distributed among the plurality of plates.

Certain embodiments disclosed herein include a method, non-transitory computer readable medium, or system as noted above or below, wherein the plurality of plates is included in a plate reader, wherein the light is emitted and the plurality of signals are measured via the plate reader.

Certain embodiments disclosed herein include a method, non-transitory computer readable medium, or system as noted above or below, wherein the plurality of vesicles is included in a vesicle solution, wherein creating the vesicle solution includes extruding the mixture after a plurality of cycles of freezing and thawing.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a flowchart illustrating a method for screening for cellular toxicity according to an embodiment.

FIG. 2 is a flowchart illustrating a method for creating a shelf stable lipid-gramicidin mixture according to an embodiment.

FIG. 3 is a flowchart illustrating a method for preparing a lipid-gramicidin mixture for freezing according to an embodiment.

FIG. 4 is an illustration of various large unilamellar vesicles (LUVs) which may be utilized in accordance with various disclosed embodiments.

FIG. 5 is a graph demonstrating a quench rate which may be achieved by mixtures according to certain implementations.

FIG. 6 is a graph demonstrating individualized normalized traces utilized to illustrate an example relationship between bilayer property changes and quench rate according to certain implementations.

FIG. 7 is a schematic diagram of a computer system according to an embodiment.

### DETAILED DESCRIPTION

In light of challenges in the art, it has been identified that there is a need for scalability. That is, some existing solutions for cellular screening, and in particular for cytotoxicity screening involving synthetic membranes, are not concerned with scalability and therefore lack motivation to scale. However, as discussed further below, improving the process in a manner that increases scalability may also allow for improving shelf stability of liposomes processed this way, therefore reducing waste and optimizing processing. Thus, the disclosed embodiments provide processes which improve shelf stability and scalability. The disclosed embodiments further describe various steps, parameters, and other components of the process which allow for realizing such shelf stability.

The various disclosed embodiments include techniques for screening. Various disclosed embodiments provide an assay for measuring parameters related to a presence, amount, or functional activity of a test substance. In an embodiment, a lipid and gramicidin are mixed and prepared for processing. The mixture is frozen and then thawed in a series of cycles before being extruded. The extruded mixture is frozen and thawed. Once thawed, the extruded mixture may be run through a desalting column in order to provide a liposome solution. The liposome solution may be stored for subsequent use such as, but not limited to, for use in screening tests. For example, the liposome solution may be diluted and then used for screening.

In a further embodiment, a lipid mixture such as, but not limited to, a mixture of 1,2-dierucoyl-sn-glycero-3-phosphocholine (DC_{22:1}PC) and the naturally occurring mixture of gramicidin from Bacillus brevis (80-85% gramicidin A), both solubilized in organic solvent, are dried down under an inert gas in order to result in a film. The film is then processed as follows. In this regard, it is noted that pre-mixing the lipid mixture, i.e., mixing the lipid and gramicidin prior to drying, allows for ensuring a stable baseline throughout experimentation from a batch created using this pre-mixing. In contrast, solutions which dry down the lipid alone prior to adding gramicidin demonstrate less stable baselines throughout experimentation.

The resultant film is placed in a desiccator overnight to remove solvents. In an embodiment, the film is rehydrated for at least 4 hours, if not overnight, with a solution containing an indicator such as a fluorophore (e.g., the fluorophore, the disodium salt of 8-aminonaphthalene-1,3,6-trisulfonic acid (ANTS)). The resultant solution is then sonicated for 1 minute in a sonicator such as, but not limited to, Branson 3510 (Danbury, CT) or other sonicator for spontaneous multilamellar liposome formation and freeze-thawed for 5 cycles of 10 minutes total each in order to increase fluorophore encapsulation yield and for unilamellar liposome formation. In an embodiment, the sonicator is a low-powered sonicator, i.e., the power of the sonicator is below a threshold. In another embodiment, the film is rehydrated for less than 4 hours of time. In a further embodiment, the film is rehydrated for between 1 and 2 hours.

Liposomes are extruded using a liposome extruder (such as, but not limited to, a LIPEX® liposome extruder) under a constant pressure of 200 pounds per square inch (psi) with an inert gas to increase homogeneity of liposome size. Such a liposome extruder may force a liposome solution including the liposomes through a filter with a predetermined pore size and may further allow for continuous, stable pressure and temperature control, which in turn leads to improved reproducibility in liposome size and impacts liposome stability and scalability. The liposomes are then frozen at −40 degrees Celsius (C) or −80 degrees C. In at least some implementations, the liposomes created through the foregoing process are viable for 6-8 weeks, as opposed to 7-10 days which would be the time period of viability for liposomes created according to some existing solutions. Consequently, the liposomes created as described herein demonstrate significantly improved viability, which in turn results in liposomes which are much more shelf stable than at least some existing solutions.

In a further embodiment, the liposomes are removed from the freezer and thawed at room temperature. The liposomes are run through a size-exclusion desalting column, for example and without limitation based on Sephdex™ G-25 resin (such as, but not limited to, a protein desalting (PD) PD-10 desalting column produced by GE Healthcare, Piscataway, NJ) to remove external fluorophore (8-Aminonaphthalene-1,3,6-Trisulfonic Acid, Disodium Salt, ANTS), other potential small molecules, contaminants, salts, and the like, and any combination thereof. The desalted solution of liposomes may be diluted as desired and can be kept at +12 degrees C. for 3 weeks, as opposed to 7-10 days for some existing solutions.

In some implementations, the disclosed liposome preparation may be performed in one day. As noted herein, such a one-day process may improve scalability of the process, i.e., by allowing for creating the liposome solution and screening in a shorter period of time than processes which are performed over the course of multiple days. In such implementations, a one-day liposome preparation process may include drying the lipid-gramicidin mixture down to create a film (i.e., a dried film), desiccating the film for 2-3 hours rather than overnight, and then rehydrating the film for 1-2 hours instead of at least 4 hours. In such implementations, the rehydrated film is sonicated and subjected to cycles of pre-extrusion freezing and thawing, for example as described herein, and then extruded. In at least some such implementations, the steps of post-extrusion freezing the liposomes at −40 degrees C. or −80 degrees C. and thawing those frozen liposomes are omitted. In other such implementations, the liposome mixture is frozen post-extrusion, and the desalting and dilution steps may therefore be omitted.

In an embodiment, lipids and gramicidin are mixed together, desiccated under vacuum, rehydrated, sonicated, freeze-thawed, extruded, and frozen post-extrusion. As compared to at least some existing solutions, which dry the lipid alone and add gramicidin only after a large unilamellar vesicles (LUV) formation, pre-mixing the lipid-gramicidin mixture prior to liposome (or LUV) formation improves baseline stability, thereby allowing for continuous data acquisition. More specifically, the improved baseline stability effectively enables the ability to freeze the liposomes, which in turn can be used to preserve the liposomes such that they remain shelf stable for longer periods of time. In particular, it is noted that this pre-mixing may, in at least some implementations, provide better equilibrium of gramicidin between vesicle leaflets. Additionally, utilizing a liposome extruder and subsequently freezing the liposome by incorporating gramicidin mixture post-extrusion allows for further improving shelf stability and scalability of the preparation. Further, using a liposome extruder reduces batch-to-batch variability such that the overall process is more reproducible between batches or operators performing the process.

Some disclosed embodiments utilize a liposome extruder such as, but not limited to, a LIPEX® extruder. During development of at least some disclosed embodiments, it was noted that use of the liposome extruder in this manner provided unexpected results which further improves the processing. Such unexpected results include that shelf stability is extended for up to 3 weeks at +12 degrees C. as opposed to 7-10 days. As noted above, this may be due to the pre-mixing as described herein providing better equilibrium of gramicidin between vesicle leaflets. Moreover, the extrusion, as disclosed, may increase liposome homogeneity with respect to, for example, but not limited to, size distribution, lamellarity, and the like, and any combination thereof, which may further provide stability. It is further noted that, when the LIPEX extruder was used to extrude the DC_{22:1}PC containing liposomes 10 times through a 0.1 μm polycarbonate filter with a Nitrogen gas tank and regulator connected to provide a constant pressure of 200 psi, liposomes were frozen after extrusion at −40 degrees C. or −80 degrees C. and were found to be viable for 6-8 weeks after post-freeze freeze-thaw. After elution over a desalting column, and dilution in a buffer (140 mM NaNO₃, 10 mM HEPES in water, pH7), liposomes were found to be shelf stable for up to 3 weeks at +12 degrees C.

Various disclosed embodiments also include techniques for fitting data. In an embodiment, fitting the data includes utilizing a Stern-Volmer corrected modified stretched exponential having replaced the previous fit (standard stretched exponential) as it allows for a) accounting for extravesicular ANTS, b) extrapolating back to t=0 and c) a non-quenchable population of vesicles. Exploiting the increased signal/noise ratio obtained with LED light sources, it further becomes possible to subtract a fluorescence signal from the molecule being investigated.

Additionally, various disclosed embodiments reduce baseline rate shifting, that is the number of membrane-spanning dimers formed in liposomes without test substance, thereby reducing the number of times a baseline must be established and enabling continuous data acquisition. Some existing solutions which may require repeatedly establishing a new control baseline throughout experimentation due to equilibrium shifting. Various disclosed embodiments provide more stable compositions and thus, larger batches of liposomes, thereby minimizing the need to reestablish fluorescence-quench control baselines.

In some embodiments, synthetic lipids (e.g., liposomes or other artificial vesicles) are utilized for the screening. In other embodiments, lipid membranes may be extracted from living cells and utilized for the screening. Moreover, in some embodiments, different synthetic membranes may be utilized as a proxy for screening different cells such as, but not limited to, cells of different organs, i.e., such that the screening may be performed based on organ-specific lipid compositions.

Various disclosed embodiments may be utilized for a variety of implementations such as, but not limited to, screening for cytotoxicity, screening for environmental toxins, for drug discovery, for identifying drugs that will be clinical trial failures (phase 1-3 and post-phase 3 such as, but not limited to, drugs that have cleared clinical trials but later need to be withdrawn), for screening cosmetic ingredients, for screening skin care ingredients, for drug delivery systems, combinations thereof, portions thereof, and the like. To this end, during screening, components or values or any of the following may be measured cytotoxicity, toxicity, clinical trial failure, cosmetic ingredients, skincare ingredients, drug delivery systems, food additives, supplements, nutraceuticals, molecules, biologics, and environment toxins. The disclosed embodiments may also be utilized for food additives, supplements, herbal remedies/medications (nutraceuticals), vitamins, combinations thereof, and the like.

In some embodiments, a screening process is used to record signals. Such a screening process may include, but is not limited to, emitting light on vesicles in order to excite the vesicles (e.g., by exciting a chemical compound such as, but not limited to, fluorophore, within the vesicles) and measuring signals of the excited vesicles. To this end, in a further embodiment, the vesicles may be distributed among multiple plates (e.g., plates of a plate reader), and the light may be emitted on the vesicles as distributed among the plates. Further, in at least some such embodiments, the vesicles each have a diameter between 100 and 400 nanometers. In yet a further embodiment, the vesicles each have a diameter of around 130 nanometers or 300 nanometers. The vesicles may be part of a vesicle solution created as discussed herein, and in particular by premixing a lipid and protein into a mixture before one or more cycles of freezing and thawing the mixture and then extruding the mixture after the cycles. As discussed herein, after extruding the mixture which has been subject to freezing and thawing cycles, the resulting extruded mixture may be frozen.

In this regard, it is noted that vesicles tend to be unstable, and that such instability makes screening vesicles using a device such as a plate reader challenging. As noted above, premixing the lipid and protein improves stability of the resulting vesicles. Consequently, screening the vesicles which are premixed as described herein may allow for screening vesicles with smaller sizes (e.g., hundreds of nanometers in diameter) than cells or other structures which are typically analyzed using a device such as a plate reader. As a result, such smaller vesicles may allow for more accurately recording signals. More specifically, various disclosed embodiments may allow for more granularly detecting membrane damage or other changes in membrane properties such as, but not limited to, aggregate changes in curvature, thickness, elasticity, and the like in real-time.

It should be noted that various disclosed embodiments are utilized for small molecules (e.g., molecules having a molecular weight below a threshold such as 1000 Daltons), but that at least some disclosed embodiments may be utilized for other types of molecules (e.g., larger molecules having a molecular weight above 1000 Daltons).

It should be noted that various disclosed embodiments are utilized for an assay for measuring the effect of a test substance on a lipid bilayer. In at least some embodiments, liposomes (or vesicles) in this process are composed of a lipid bilayer, a pair of an indicator and a quencher (such as, but not limited to, a monovalent cation), and a protein (e.g., a transmembrane protein such as gramicidin), wherein the vesicles encompass one member of the pair of the indicator and the monovalent cation, and comprise gramicidin in the lipid bilayer. In various embodiments, the lipid bilayer is formed using lipids (such as, but not limited to, phospholipids, sterols, glycolipids, etc.) selected in order to increase membrane stiffness, increase membrane elasticity or softness, increase or decrease membrane thickness, increase or decrease membrane curvature, allow for gain/loss of gramicidin function, a combination thereof, and the like.

In a further embodiment, the lipid bilayer is formed at least using phospholipids selected from the group consisting of di-C_{22:1}, di-C_{20:1}-, di-C_{18:1}-, -1-C₁₆, -2C_{18:1}-acyl chains2-Cs.1-acyl chains, and combinations thereof, in the absence or presence of cholesterol and/or other lipids. In yet a further embodiment, the lipid bilayer is formed using phospholipids selected from the group consisting of di-C_{22:1}, di-C_{20:1}-, di-C_{18:1}-, -1-C₁₆, -2C_{18:1}-acyl chains2-Cs.1-acyl chains, and combinations thereof, in the absence or presence of cholesterol and/or other lipids. In an embodiment, molecules of gramicidin present in the lipid bilayer are in a monomer dimer equilibrium in favor of monomer. Non-limiting example compositions for liposomes which may be utilized in accordance with various disclosed embodiments are described further in U.S. Pat. No. 8,901,042, the contents of which are hereby incorporated by reference.

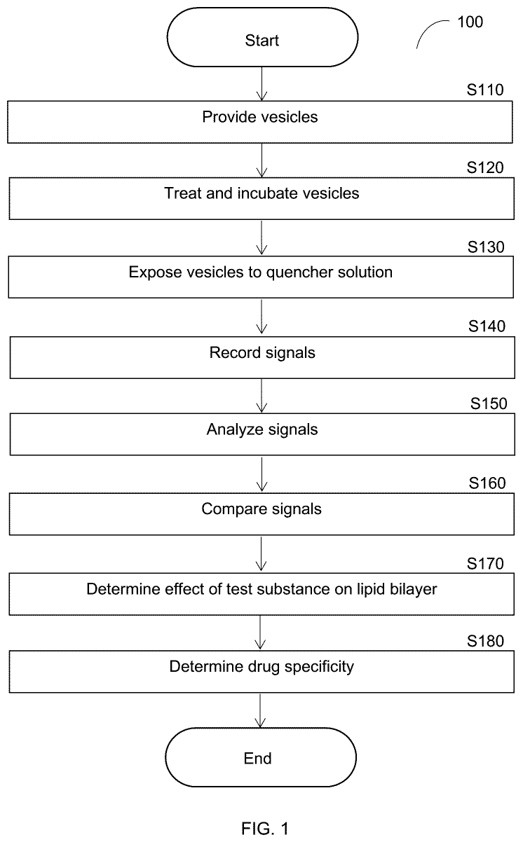

FIG. 1 is a flowchart 100 illustrating a method for screening for cellular toxicity according to an embodiment.

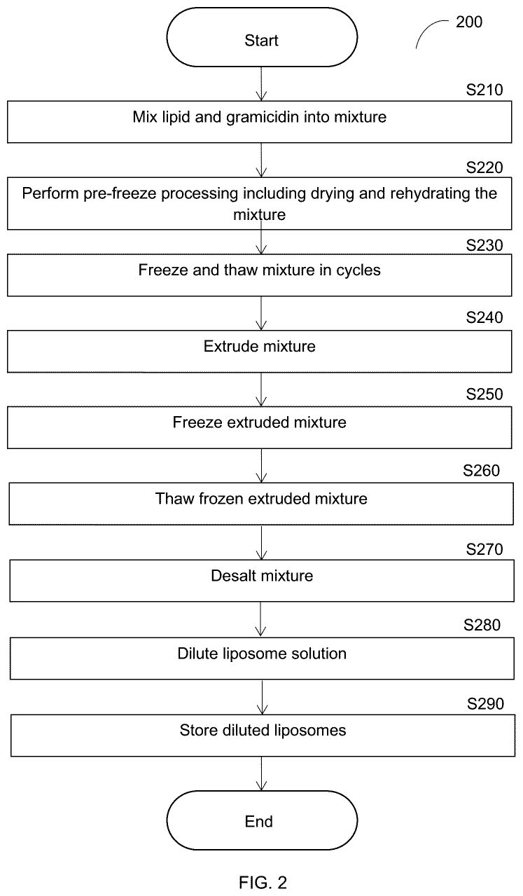

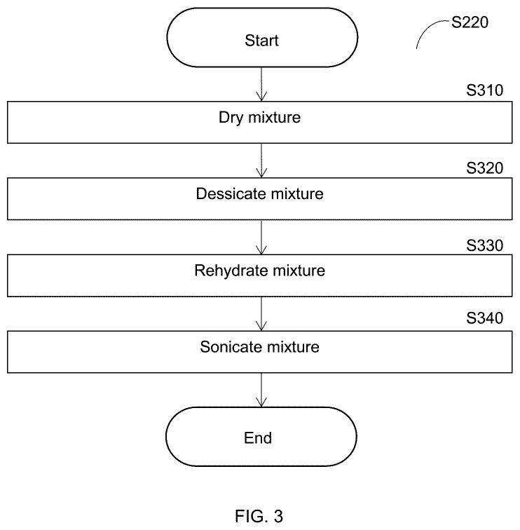

At S110, vesicles are provided. In an embodiment, the vesicles are or include liposomes created as described herein, for example as described with respect to FIGS. 2-3.

At S120, the vesicles are treated and incubated. In an embodiment, the vesicles are treated with a solution of test substances at a predetermined concentration and incubated. In such an embodiment, during incubation, the test substances are allowed to interact with the vesicles in the vesicle-test substance mixture and reach adsorption equilibrium. In an embodiment, no test substances (i.e., a solution of 0 μm test substance) may be added for treatment and incubation of vesicles.

At S130, the vesicles are exposed to a quencher solution. In an embodiment, the quencher solution includes gramicidin channel permeable cations. In a further embodiment, the quencher solution includes a monovalent cation of a pair of an indicator and the monovalent cation. In an example embodiment, the pair of the indicator and the monovalent cation may be a pair of disodium salt of 8-aminonaphthalene-1,3,6-trisulfonic acid (ANTS) and Tl+.

At S140, signals are recorded for the vesicles. In an embodiment, the recorded signals are fluorescence signals recorded of the fluorophores provided in the vesicles.

In an embodiment, a screening process is used to record the signals. Such a screening process may include, but is not limited to, emitting light on vesicles in order to excite the vesicles (e.g., by exciting one or more chemical compounds within the vesicles) and measuring signals of the excited vesicles. To this end, in a further embodiment, the vesicles may be distributed among multiple plates (e.g., plates of a plate reader), and the light may be emitted on the vesicles as distributed among the plates. Further, in at least some such embodiments, the vesicles each have a diameter between 100 and 400 nanometers. In yet a further embodiment, the vesicles each have a diameter of around 130 nanometers or 300 nanometers. The vesicles may be part of a vesicle solution created as discussed herein, and in particular by premixing a lipid and protein into a mixture before one or more cycles of freezing and thawing the mixture and then extruding the mixture after the cycles.

At S150, the recorded signals are analyzed.

At S160, the signals are compared. In an embodiment, the signals are compared to a baseline of the vesicles of the same batch which are not exposed to the solution of a test substance. In a further embodiment, the signals may be compared to recorded signals of vesicle samples that are treated to solutions with different concentrations of the test substance. It should be noted that the vesicles prepared as described herein provide a stable baseline with less baseline shifting. Furthermore, the scalable property eliminates repeated measurement of the baseline thereby reducing uncertainties and increasing accuracy of the assay.

At S170, an effect of the test substance on the lipid bilayers of the vesicles is determined. A quench rate of the fluorescence signal may be determined which may indicate a property change (or lack thereof) in the lipid bilayer. In an example implementation, a high quench rate (e.g., above a predetermined threshold) indicates a large change of the lipid bilayer (membrane property) due to the test substance. In a further example implementation, a low (e.g., below a predetermined threshold) quench rate indicates a smaller change in the lipid bilayer of the vesicle. The large effect of the test substance on the vesicles (i.e., as evidenced by a high quench rate in such an example implementation) may indicate higher toxicity of the test substance on the lipid bilayer, and thus, greater cellular toxicity. In other implementations, high or low quench rates may correspond to other kinds of property changes in the vesicle membrane.

In some embodiments, the recorded signals are analyzed and the effect of the test substance is determined after a period of time of exposure to the test substance. In a further embodiment, a dose-response relationship is determined for the period of exposure time based on the effect of the test substance. The dose-response relationship represents a magnitude of the response of an organism as a function of exposure to a stimulus (i.e., the test substance) after a period of exposure time. More specifically, in an embodiment, different concentrations of the test substance (e.g., different dosages of a drug acting as the test substance) may be tested, and responses (e.g., a response such as an increase in fluorescence quench rate) to the different concentrations are recorded. Based on the recorded responses and the concentrations, a dose-response relationship may be determined for the test substance. More specifically, in at least some implementations, exposure to the test substance may cause changes in membrane properties (e.g., properties measured as real-time aggregate changes in curvature, elasticity, thickness, etc.) which cause an increase in activity of a reporter protein (e.g., gramicidin). Such increased reporter protein activity may be indicative of, for example, an increase in the conduction of Thallium from the quencher solution, thereby demonstrating an increased fluorescence quench rate. Such a dose-response relationship may be utilized, for example, to determine an optimal or otherwise suitable dosage of a drug.

In an embodiment, the effect of the test substance is or includes one or more changes in membrane properties of the lipid bilayers caused by exposure to the test substance. In another embodiment, the effect of the test substance is or includes a lack of change in membrane properties of the lipid bilayers as a result of exposure to the test substance. Such changes in membrane properties may be utilized, for example, in order to determine changes in membrane properties which may be advantageous for a given drug (e.g., in order to solve a particular issue or otherwise to determine if the drug is viable for a given purpose). To this end, in a further embodiment, S170 may further include determining a viability of a drug candidate for one or more purposes based on the determined changes in membrane properties. In yet a further embodiment, the determined membrane property changes may be checked against one or more predetermined desired membrane property changes in order to determine if the effects of the test substance cause the desired membrane property changes.

At optional S180, a drug specificity may be determined based on the determined effect of the test substance. That is, when the test substance is a drug, the test used to determine the effect of the test substance may be analyzed in order to determine how the specificity of the drug. That is, the drug specificity represents whether the drug is interacting with one or more intended targets as contrasted with non-target components. In an embodiment, interactions with the vesicle membranes may be measured when the target is a target other than the membrane of each vesicle, and the specificity may be determined based on whether or to what degree off-target effects are observed (e.g., off-target effects including effects on the membrane when the membrane is not the target).

FIG. 2 is a flowchart 200 illustrating a method for creating shelf stable liposomes using a lipid-gramicidin mixture according to an embodiment.

At S210, a lipid and gramicidin are mixed into a lipid-gramicidin mixture. In an embodiment, the mixed lipid and gramicidin are solubilized in an organic solvent. In a further embodiment, the lipid and gramicidin are initially in chloroform and methanol solutions, respectively. The lipid may be in the form of a lipid mixture such as, but is not limited to, a mixture of 1,2-dierucoyl-sn-glycero-3-phosphocholine (DC_{22:1}PC). The gramicidin (gA) may be in the form of a gramicidin mixture such as, but not limited to, the naturally occurring mixture of gramicidin from Bacillus brevis (80-85% gramicidin A).

At S220, one or more pre-freeze processing steps including drying the mixture are performed. As noted above, by first mixing the lipid and gramicidin and then drying the mixture instead of drying the lipid in isolation before mixing with gramicidin, the resulting gA-liposome exhibits improved baseline stability.

In an embodiment, the pre-freeze processing steps include the steps as now described with respect to FIG. 3. FIG. 3 is a flowchart S220 illustrating a method for preparing a lipid-gramicidin mixture for freezing according to an embodiment.

At S310, the mixture is dried. In an embodiment, the mixture is dried down under Nitrogen or another inert gas in order to create an initial thin film of lipid, protein, or both. Then, chloroform begins to evaporate.

At S320, the mixture is desiccated. In an embodiment, the mixture is desiccated under vacuum for at least 4 hours. In a further embodiment, the mixture is desiccated overnight. The result may be full evaporation of the chloroform and methanol of the initial solutions that were mixed.

In another embodiment, the mixture is desiccated for 2 to 3 hours. Such a shorter desiccation period (e.g., as compared to at least 4 hours) may be utilized in an alternate embodiment that supports creating the liposomes in the process according to FIG. 2 within one day.

At S330, the mixture is rehydrated. The mixture is rehydrated in a filling buffer such as, but not limited to, 150 mM NaNO3, 15 mM HEPES, pH 7.0 in water. In an embodiment, the mixture is rehydrated for at least 1 hour. In a further embodiment, the mixture is rehydrated for at least 4 hours. In a further embodiment, the mixture is rehydrated with a solution containing the fluorophore such as, but not limited to, the disodium salt of 8-aminonaphthalene-1,3,6-trisulfonic acid (ANTS).

In the alternative embodiment where the liposomes are created within one day, the mixture is rehydrated for 1-2 hours.

At S340, the mixture is sonicated. In an embodiment, the mixture is sonicated for 1 minute at room temperature.

Returning to FIG. 2, at S230, the mixture is frozen and thawed in a series of cycles. In an embodiment, the mixture is frozen and thawed in 5 cycles. In a further embodiment, the mixture is frozen for 5 minutes on dry ice and thawed for 5 minutes in warm water (e.g., above a predetermined threshold temperature) for a total of 10 minutes in each cycle.

At S240, the mixture is extruded. In an embodiment, S240 includes using a liposome extruder. As noted above, such a liposome extruder has demonstrated further improvements to stability of the resulting liposomes.

At optional S250, the extruded mixture is frozen. In an embodiment, the mixture is frozen for storage at −40 degrees C. or at −80 degrees C. In a further embodiment, the components of the extruded mixture are viable for 6 to 8 weeks when stored at −40 degrees C. or at −80 degrees C.

In the embodiment where the liposomes are created within one day, step S250 may optionally be omitted and the mixture is not frozen at −40 degrees C. or at −80 degrees C.

At S260, the frozen extruded mixture is thawed. In an embodiment, the mixture is thawed at room temperature.

At optional S270, the mixture may be desalted. In an embodiment, S270 includes running the mixture through a desalting column such as, but not limited to, a desalting column. In a further embodiment the mixture is run through the desalting column with a buffer such as, but not limited to, 140 mM NaNO3, 10 mM HEPES, pH 7.0 in water. The mixture may be desalted in order to remove extravesicular fluorophore (e.g., 8-Aminonaphthalene-1,3,6-Trisulfonic Acid, Disodium Salt, ANTS) and multilamellar liposomes. To this end, the throughput liposome solution of the desalting column may include homogenous or relatively homogeneous gA-liposomes.

At S280, the liposome solution may be diluted. The liposome solution may be diluted in aqueous solutions such as, but not limited to, 140 mM NaNO₃, 10 mM HEPES, pH 7.0 in water, for utilization in, for example, toxicity screening.

At S290, the diluted liposomes are stored for subsequent use (e.g., for use in an assay such as, but not limited to, as described above with respect to FIG. 1). In an embodiment, the diluted liposomes are stored at a temperature, for example but not limited to, a temperature which is between transition temperatures of the lipid such as around 12 degrees C. for DC_{22:1}PC. It should be noted that other transition temperature values may be utilized for certain other lipids. In a further embodiment, the diluted liposomes are stored at a temperature between a gel transition temperature and a liquid crystalline transition temperature for the lipid. In yet a further embodiment, the diluted liposomes are stored at a temperature in the range of +7 to +13 degrees C. According to certain embodiments, the diluted liposomes may be stored for up to 3 weeks while maintaining baseline stability as compared to at least some existing solutions, which only support storing the diluted liposomes for around 7-10 days.

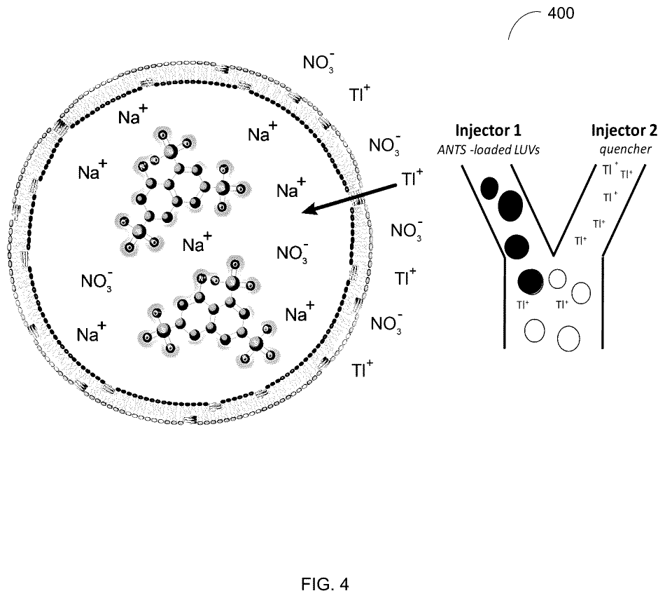

FIG. 4 is a non-limiting example illustration 400 of various large unilamellar vesicles (LUVs) which may be utilized in accordance with various disclosed embodiments.

In an experiment whose results are depicted in the non-limiting example illustration 400, LUVs composed of 1,2-dierucoyl-sn-glycero-3-phosphocholine (DC_{22:1}PC) and gramicidin from Bacillus brevis were dried down under nitrogen, desiccated overnight, and prepared through a four-part process of sonication, freeze-thaw cycles, extrusion, and elution over a desalting column. The fluorescence quench rate is measured by combining the ANTS filled LUVs (Black circles) with a Tl+ quencher solution (50 mM titanium (IV) nitrate (TINO3), 94 mM sodium nitrate (NaNO3), and 10 mM 4-(2-hydroxyethyl)-1-piperazineethanesulfonic acid (HEPES) at pH 7.0) in a stopped-flow spectrofluorometer (Applied Photophysics, SX20, Leatherhead, England).

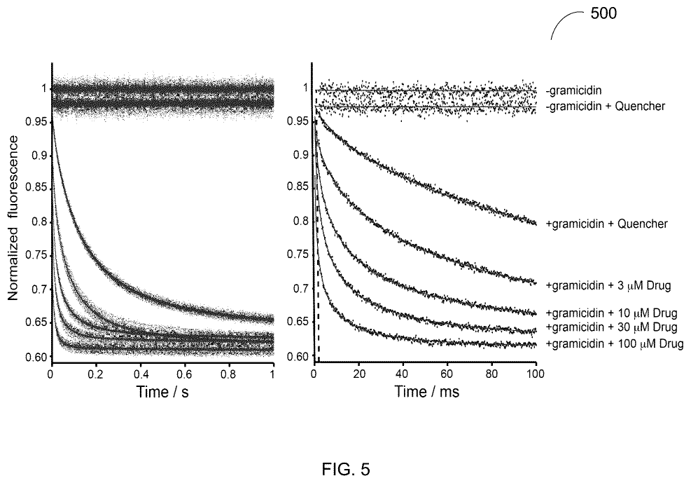

FIG. 5 is a non-limiting example graph 500 demonstrating a quench rate which may be achieved by mixtures according to certain implementations.

As shown in the graph 500, the quench rate is determined by fitting to the first 2-100 millisecond (ms) of the individual quenching curves, and the normalized data is shown. Fluorescence quench traces recorded both in the absence and presence of gramicidin at different concentrations of a bilayer-modifying drug are depicted. The left panel shows 1 second(s) traces. In the left panel, each line illustrates an averaged trace for each experimental condition, where the dots surrounding each line indicate results from all repeats. In the right panel, which shows the first 100 ms of quenching, the dots surrounding each line indicate results from a single repeat and each line shows the fit of the stretched exponential to those repeats. In an embodiment, the time of 2 ms, the rate of quenching is determined. This may be performed, for example, when the quench rate is determined pursuant to a standard stretched exponential function. In another embodiment, the rate of quenching may be extrapolated back to a time of t=0 ms. This may be performed, for example, when the quench rate is determined pursuant to a Stern-Volmer corrected modified stretched exponential function.

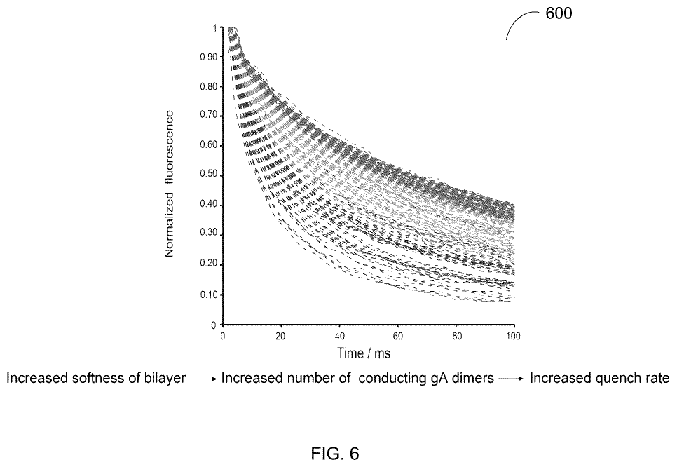

FIG. 6 is a non-limiting example graph 600 demonstrating individualized normalized traces utilized to illustrate an example relationship between changes in bilayer properties and quench rate according to certain implementations.

As shown in the graph 600, the time course of fluorescence by the relative fluorescence decreases. A rapid decrease in fluorescence is shown with increasing concentration of the bilayer-modifying drug. As the bilayer is perturbed, generally becoming softer, the overall gramicidin activity increases, which results in increased quench rates.

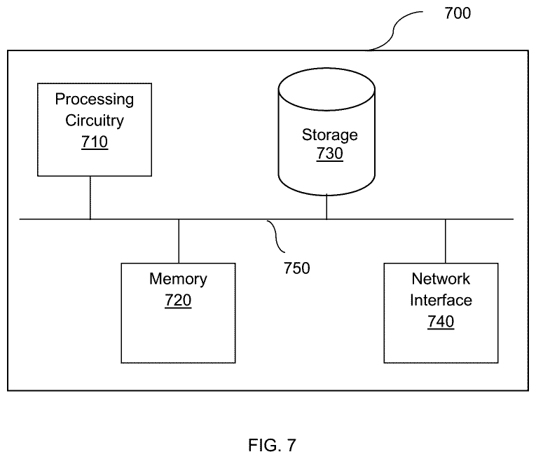

FIG. 7 is an example schematic diagram of a hardware layer 700 according to an embodiment. The hardware layer 700 may be utilized to realize at least a portion of at least some disclosed embodiments. In particular, fits to be utilized in accordance with various disclosed embodiments may be determined via software executed via the hardware layer 700. In embodiments where at least a portion of the process is automated or performed via one or more robotic devices (e.g., a robotic device having parts to aid in mixing, freezing, measuring, etc.), the hardware layer 700 may be utilized to run software for controlling the process, determining effects of test substances on lipid bilayers (e.g., including but not limited to calculating values indicative of such effects), or otherwise manipulating such robotic devices.

As depicted in FIG. 7, the hardware layer 700 includes a processing circuitry 710 coupled to a memory 720, a storage 730, and a network interface 740. In an embodiment, the components of the hardware layer 700 may be communicatively connected via a bus 750.

The processing circuitry 710 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information.

The memory 720 may be volatile (e.g., random access memory, etc.), non-volatile (e.g., read only memory, flash memory, etc.), or a combination thereof.

In one configuration, software for implementing one or more embodiments disclosed herein may be stored in the storage 730. In another configuration, the memory 720 is configured to store such software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the processing circuitry 710, cause the processing circuitry 710 to perform the various processes described herein.

The storage 730 may be magnetic storage, optical storage, and the like, and may be realized, for example, as flash memory or other memory technology, compact disk-read only memory (CD-ROM), Digital Versatile Disks (DVDs), or any other medium which can be used to store the desired information.

The network interface 740 may allow the hardware layer 700 to communicate with devices of remote operators, control systems, and the like.

It should be understood that the embodiments described herein are not limited to the specific architecture illustrated in FIG. 7, and other architectures may be equally used without departing from the scope of the disclosed embodiments.

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

At least some embodiments disclosed herein can be implemented as hardware, firmware, software, or any combination thereof. Moreover, the software may be implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for solution preparation, comprising:
mixing a lipid and a protein to create a mixture;
freezing and thawing the mixture in a plurality of cycles;
extruding the mixture after the plurality of cycles; and
producing a vesicle solution using the extruded mixture.

2. The method of claim 1, wherein the protein is a transmembrane protein.

3. The method of claim 1, wherein the protein is gramicidin.

4. The method of claim 1, further comprising:
freezing the extruded mixture; and
thawing the frozen extruded mixture, wherein the vesicle solution is produced using the extruded mixture when the extruded mixture has been frozen and thawed.

5. The method of claim 1, wherein producing the vesicle solution further comprises:
running the extruded mixture through a desalting column.

6. The method of claim 1, wherein mixing the lipid and gramicidin further comprises:
mixing a lipid solubilized in a first organic solvent and a gramicidin solubilized in a second organic solvent to create a film; and
drying the film, where the mixture includes the film after the film has been dried.

7. The method of claim 6, further comprising:
rehydrating the film when the film has been dried, wherein the mixture includes the film which has been rehydrated.

8. The method of claim 7, wherein the film is rehydrated for at least 4 hours.

9. The method of claim 7, wherein the film is rehydrated for between 1 and 2 hours.

10. The method of claim 1, further comprising:
screening, using the vesicle solution, for at least one of cytotoxicity, toxicity, clinical trial failure, cosmetic ingredients, skincare ingredients, drug delivery systems, food additives, supplements, nutraceuticals, molecules, biologics, and environment toxins.

11. The method of claim 1, wherein the lipid is a lipid mixture of 1,2-dierucoyl-sn-glycero-3-phosphocholine (DC_{22:1}PC).

12. The method of claim 1, wherein vesicle solution includes a gramicidin mixture of between 80 and 85 percent gramicidin A.

13. The method of claim 1, further comprising:
treating the vesicle solution using a test substance;
incubating the vesicle solution which has been treated with the test substance;
exposing the vesicle solution to a quencher solution; and
recording a plurality of signals for the vesicle solution.

14. The method of claim 13, wherein the test substance is a drug, further comprising:
determining a specificity of the drug based on the recorded signals.

15. The method of claim 13, wherein the plurality of signals is recorded after a period of exposure time, further comprising:
determining a dose-response relationship for the period of exposure time.

16. The method of claim 13, wherein the quencher solution includes gramicidin channel permeable cations.

17. The method of claim 16, wherein the quencher solution includes a monovalent cation of a pair of an indicator and the monovalent cation.

18. The method of claim 13, wherein the vesicle solution includes a plurality of vesicles, further comprising:
analyzing the plurality of signals; and
determining an effect of the test substance on lipid bilayers of the plurality of vesicles of the vesicle solution.

19. The method of claim 18, wherein each of the vesicles has a diameter between 100 and 400 nanometers.

20. The method of claim 18, wherein each of the vesicles has a diameter of 130 nanometers.

21. The method of claim 18, wherein each of the vesicles has a diameter of 300 nanometers.

22. The method of claim 18, wherein the determined effect of the test substance includes at least one change in membrane property.

23. The method of claim 22, further comprising:
determining a viability of a drug candidate for at least one purpose based on at least one change in membrane property.

24. The method of claim 18, wherein the determined effect of the test substance includes a lack of change in membrane properties.

25. A method for screening, including:
treating a plurality of vesicles using a solution of at least one test substance, wherein at least a portion of the plurality of vesicles is created by at least premixing a lipid and a protein into a mixture before freezing and thawing the mixture;
incubating the plurality of vesicles;
exposing the plurality of vesicles to a quencher solution; and
recording a plurality of signals for the plurality of vesicles.

26. The method of claim 25, wherein the quencher solution includes gramicidin channel permeable cations.

27. The method of claim 26, wherein the quencher solution includes a monovalent cation of a pair of an indicator and the monovalent cation.

28. The method of claim 25, wherein the plurality of signals is a plurality of fluorescence signals of fluorophores in the plurality of vesicles.

29. The method of claim 25, wherein the plurality of vesicles is a first plurality of vesicles included in a batch, wherein the batch includes the first plurality of vesicles and a second plurality of vesicles, further comprising:
comparing the plurality of signals to a baseline of the second plurality of vesicles.

30. The method of claim 29, wherein the second plurality of vesicles is not exposed to the test substance.

31. The method of claim 29, wherein the second plurality of vesicles is treated using a different concentration of the test substance than the first plurality of vesicles.

32. The method of claim 29, further comprising:
determining an effect of the test substance on the first plurality of vesicles based on the plurality of signals.

33. The method of claim 25, wherein the plurality of vesicles is from a vesicle solution produced using a lipid-gramicidin mixture.

34. The method of claim 23, wherein the lipid-gramicidin mixture is produced using a process including premixing lipid and gramicidin into a film before drying the film to produce a dried film.

35. The method of claim 34, wherein the dried film is rehydrated, wherein the lipid-gramicidin mixture includes the rehydrated film.

36. The method of claim 25, wherein the plurality of vesicles is treated using the solution of the test substance at a predetermined concentration.

37. The method of claim 25, wherein each of the vesicles has a diameter between 100 and 400 nanometers.

38. The method of claim 25, wherein each of the vesicles has a diameter of 130 nanometers.

39. The method of claim 25, wherein each of the vesicles has a diameter of 300 nanometers.

40. The method of claim 37, wherein recording the plurality of signals for the plurality of vesicles further comprises:
emitting light on the plurality of vesicles in order to excite the plurality of vesicles; and
measuring the plurality of signals based on the excited vesicles.

41. The method of claim 40, wherein recording the plurality of signals for the plurality of vesicles further comprises:
distributing the plurality of vesicles among a plurality of plates, wherein the light is emitted on the plurality of vesicles distributed among the plurality of plates.

42. The method of claim 41, wherein the plurality of plates is included in a plate reader, wherein the light is emitted and the plurality of signals are measured via the plate reader.

43. The method of claim 25, wherein the plurality of vesicles is included in a vesicle solution, wherein creating the vesicle solution includes extruding the mixture after a plurality of cycles of freezing and thawing.
